(19) United States
(12) Patent Application Publication
(10) Pub. No.: US 2007/0191729 A1
(43) Pub. Date: Aug. 16, 2007

(54) Portable device for user's basal body temperature and method for operating the device

(75) Inventors: Jeong Je Park, Yongin-si (KR); Kwang Hyeon Lee, Yongin-si (KR); Hye Jin Jung, Seoul (KR); Kyung Ho Kim, Yongin-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/480,858

(22) Filed: Jul. 6, 2006

(30) Foreign Application Priority Data
Feb. 16, 2006 (KR) 10-2006-0015318

(51) Int. Cl. A61B 10/00; A61B 5/00

(52) U.S. Cl. 600/551; 600/549

(57) ABSTRACT

A basal body temperature (BBT) measurement method includes: measuring each distance to at least one ear canal area by emitting a pulse to a user's ear canal; measuring a body temperature in the at least one ear canal area utilizing infrared rays; recognizing a first body temperature corresponding to a first distance which satisfies a predetermined standard, among the each distance to the at least one ear canal area, as the user's eardrum temperature; and converting the eardrum temperature into the user's BBT.

### CROSS-REFERENCE TO RELATED APPLICATION

This application claims the benefit of Korean Patent Application No. 10-2006-0015318, filed on Feb. 16, 2006, in the Korean Intellectual Property Office, the disclosure of which is incorporated herein by reference.

### BACKGROUND OF THE INVENTION

1. Field of the Invention

The present invention relates to a portable device for measuring a user's basal body temperature (BBT) and a BBT measurement method utilizing the portable device. More particularly, the present invention relates to a portable BBT measurement device and method which can detect a location of a user's eardrum via a predetermined distance sensor, measure the user's eardrum temperature, convert the eardrum temperature into the user's BBT and record the converted BBT into a memory, to generate the user's menstruation information from the BBT recorded for a certain period, e.g. a month, and provide the user with the generated menstruation information.

2. Description of Related Art

Currently, as ubiquitous related technologies receive great attention, ubiquitous technologies utilizing a portable device are also being rapidly developed. In particular, Ubiquitous-HealthCare (hereinafter, U-HealthCare) has recently been in the spotlight as a notable technology area due to the “well-being” boom. U-HealthCare means ubiquitous technology which enables anyone to readily receive medical services at any time and at any place by installing medical service-related chips or sensors in places of the user's living space. With U-HealthCare, various types of medical attention, such as physical examinations, disease management, emergency care, consultation with a doctor and the like, which currently are only performed in hospitals, may be naturally integrated into user's daily lives, and thus may be accomplished without going to a hospital.

Many diseases are generally the result of stress which is caused by busy daily lives. Particularly, for women, the stress may cause a change in an ovulation phase. In modern society where an individual's sexual lifestyle is more liberalized, women need to accurately know their ovulation phase. When women are fully aware of their own ovulation day accurately, women may more easily recognize their own menstruation information, such as a fertile period or an infertile period. The ovulation phase information may also provide information on various types of women's diseases.

However, as described above, the ovulation day of a woman who used to have normal menstruation may also be easily changed due to various reasons, e.g. stress. In this case, since the ovulation day becomes irregular, the woman may not accurately be aware of her fertile period or infertile period based on the ovulation day information that she previously knew.

Accordingly, the ovulation day needs to be measured so as to accurately understand women's frequently changing menstruation period. Measurement methods of the ovulation day include a method of checking luteinizing hormone (LH) via urine, an LH measurement method of measuring the concentration of a progesterone, an ovulation ultrasonic inspection method of measuring the size of an ovarian follicle, and a BBT measurement method utilizing the principle that a BBT rises by about 0.3 to about 0.6 degrees after ovulation.

The BBT measurement method is being widely utilized as the ovulation day measurement method due to its convenience. A BBT is a body temperature which is measured when a person is in a stable state. A body temperature when waking up in the morning after having a sound sleep may be set as the BBT. Accordingly, women may understand their own ovulation day by taking their body temperature after waking up in the morning.

FIG. 1 is a graph illustrating a correlation between a woman's ovulation day and her BBT.

As shown in FIG. 1, when the woman's menstruation period is set between 28 to 30 days, the days may be divided into a low temperature period and a high temperature period, based on the ovulation day. In this instance, the low temperature day indicates around about 14 days from a first day of menstruation to the ovulation day. During the low temperature period, the woman's temperature falls and the endometrium gets thicker due to acts of ovarian follicle hormones to be ready to receive a fertilized egg. A body temperature may significantly fall for a certain period before changing from the low temperature period to the high temperature period. The woman ovulates at any time between the certain period and two or three days before the high temperature period. The ovarian follicle after the ovulation becomes corpus luteum and LH is produced, which makes the endometrium softer so that an egg may be easily implanted. During this period, the body temperature rises.

As described above, the BBT is divided into the low temperature period and the high temperature period based on the ovulation day, and the body temperature repeats rising and falling. When menstruation starts, a body temperature is maintained at a low temperature for about two weeks and repeats rising and falling within about 0.1 degree. During the period, follicle hormones are produced from hypophyses beneath the cerebrum and an ovarian follicle in the ovary starts growing, estradiol is produced from the ovary and the endometrium gets thicker. Accordingly, as shown in FIG. 1, the fertile period may correspond to 7 days before the ovulation day and 4 days after the ovulation day.

Accordingly, when a woman takes and records her temperature for a predetermined period, she may know her ovulation rhythms. Also, she may know the ovulation day based on the BBT rhythms and also check whether the ovulation has occurred, whether the growth of an ovarian follicle or a function of an egg cell is normal, whether an activity of a corpus luteum hormone is normal, etc., from the body temperature graph.

As described above, the measurement of menstruation information according to the BBT measurement is very important to women. However, it may be very difficult for women to take their BBT every morning due to their own busy daily lives. Also, since people may not sleep soundly due to excessive job tasks or studies, even though they may take their temperature in the morning, it may not be regarded as an accurate BBT.

Accordingly, there is a demand for the development of a portable device which women living in the modern society can easily utilize without regard to a time and a place to take their temperature, and in this case, can convert the temperature into a BBT, generate menstruation information and also provide the user with the generated menstruation information, so that the user may more easily know her own menstruation information.

### BRIEF SUMMARY

An aspect of the present invention provides a Portable BBT measurement device and method which can convert a measured body temperature into a BBT in consideration of a user's current state and measurement time, so that the user may easily measure the user's BBT without regard to a time and a place.

An aspect of the present invention also provides a portable BBT measurement device and method which can detect a location of an eardrum of a user via a distance sensor, such as an ultrasonic sensor, a laser sensor, etc., measure a temperature of the detected eardrum via an infrared ray temperature sensor, so that the user may measure the user's internal temperature without regard to a change in external environments.

An aspect of the present invention also provides a portable BBT measurement device and method which enables a user of the portable device to easily take the user's temperature while the user is making a call by installing a distance sensor and an infrared ray temperature sensor around a speaker of the portable device.

An aspect of the present invention also provides a portable BBT measurement device and method which can record a user's measured BBT in a predetermined memory, generate the user's fertile period information, infertile period information, ovulation day information, or menstruation period information from the BBT that is recorded for a predetermined period, e.g. a month, and provide the user with the generated menstruation information, so that the user may accurately know her own menstruation information with only a measurement of a body temperature.

An aspect of the present invention also provides a portable BBT measurement device and method which can protect a user's privacy by measuring the user's temperature and generating the menstruation information only when the user is authenticated as a subject for measurement of a BBT via speech recognition or when a selection is received from the user.

According to an aspect of the present invention, there is provided a BBT measurement method including: measuring at least one distance to at least one ear canal area; measuring a body temperature in the at least one ear canal area; recognizing a first body temperature corresponding to a first distance which satisfies a predetermined standard, among the measured at least one distance to the at least one ear canal area, as the user's eardrum temperature; and converting the eardrum temperature into the user's basal body temperature.

According to another aspect of the present invention, there is provided a BBT measurement method including: measuring a user's body temperature; reading a time when the body temperature is measured; and converting the measured body temperature into the basal body temperature based on the user's body temperature rise value according to the read time.

According to still another aspect of the present invention, there is provided a BBT measurement method including: measuring a user's body temperature; reading the user's activity state at a time when the body temperature is measured; and converting the measured body temperature into the basal body temperature based on a predetermined body temperature calibration value according to the read activity state.

According to another aspect of the present invention, there is provided a method of measuring a basal body temperature, including: measuring at least one distance to at least one ear canal area; measuring a body temperature in the at least one ear canal area; recording the measured at least one distance and the measured body temperature in a memory, to correspond to each other; recognizing a first body temperature which is measured in correspondence to a first distance satisfying a predetermined standard, among the measured at least one distance to the at least one ear canal area, as the user's eardrum temperature; converting the eardrum temperature into the basal body temperature based on either the user's body temperature rise value according to a time when the eardrum temperature is measured or a predetermined body temperature calibration value according to the user's activity state at the time when the eardrum temperature is measured, and recording the converted basal body temperature into the memory; and generating the user's menstruation information from at least one basal body temperature which is recorded in the memory for a predetermined period and controlling the generated menstruation information to be displayed or played for the user.

According to yet another embodiment of the present invention, there is provided a BBT measurement device including: a distance sensor measuring at least one distance to at least one ear canal area by emitting a pulse to a user's ear canal; an infrared ray temperature sensor measuring a body temperature in the at least one ear canal area utilizing infrared rays; a data control unit recording the measured at least one distance and the measured body temperature in a memory, to correspond to each other, and recognizing a first body temperature which is measured in correspondence to a first distance satisfying a predetermined standard, among the at least one distance to the at least one ear canal area, as the user's eardrum temperature; a basal body temperature converter converting the eardrum temperature into the basal body temperature based on either the user's body temperature rise value according to a time when the eardrum temperature is measured or a predetermined body temperature calibration value according to the user's activity state at the time when the eardrum temperature is measured, and recording the converted basal body temperature into the memory; and an information control unit generating the user's menstruation information from at least one basal body temperature which is recorded in the memory for a predetermined period and controlling the generated menstruation information to be displayed or played for the user via a display unit or an audio output unit.

According to another aspect of the present invention, there is provided an apparatus for measuring a basal body temperature, including: a distance sensor measuring distances to ear canal areas; a temperature sensor measuring body temperatures in the ear canal areas corresponding to the measured distances; determining a body temperature corresponding to a first one of the measured distances satisfying set criteria, as the user's eardrum temperature; and converting the eardrum temperature into the basal body temperature.

According to other aspects of the present invention, there are provided computer-readable recording media storing programs for executing the aforementioned methods.

Additional and/or other aspects and advantages of the present invention will be set forth in part in the description which follows and, in part, will be obvious from the description, or may be learned by practice of the invention.

### BRIEF DESCRIPTION OF THE DRAWINGS

The above and/or other aspects and advantages of the present invention will become apparent and more readily appreciated from the following detailed description, taken in conjunction with the accompanying drawings of which:

FIG. 1 is a graph illustrating a correlation between a women's ovulation day and BBT;

FIG. 2 is a block diagram illustrating a configuration of a portable BBT measurement device according to an embodiment of the present invention;

FIG. 3 is a view illustrating a user's eardrum and three distance sensors measuring an eardrum temperature according to a first example of an embodiment of the present invention;

FIG. 4 is a table illustrating an example of an eardrum temperature table according to the first example;

FIG. 5 is a view illustrating a user's eardrum and a single distance sensor measuring an eardrum temperature according to a second example of an embodiment of the present invention;

FIG. 6 is a table illustrating an example of an eardrum temperature table according to the second example;

FIG. 7 is a diagram illustrating an example of a user's menstruation information displayed on a display unit of a portable device according to an embodiment of the present invention; and

FIG. 8 is a flowchart illustrating a BBT measurement method utilizing a portable device according to an embodiment of the present invention.

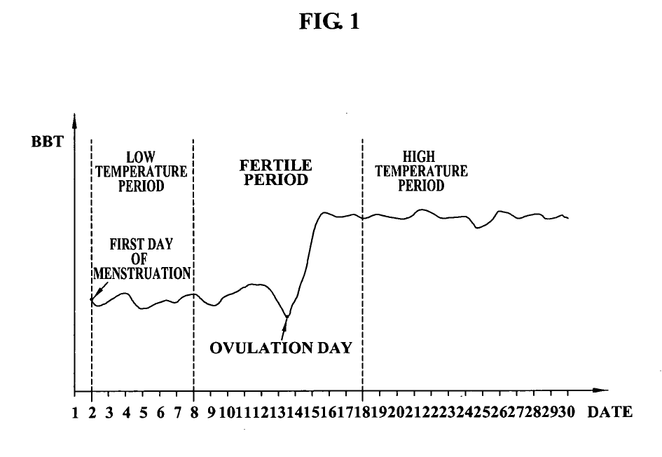

### DETAILED DESCRIPTION OF EMBODIMENTS

Reference will now be made in detail to embodiments of the present invention, examples of which are illustrated in the accompanying drawings, wherein like reference numerals refer to the like elements throughout. The embodiments are described below in order to explain the present invention by referring to the figures.

A basal body temperature (BBT) measurement method according to an embodiment of the present invention may be practiced using a predetermined BBT measurement device. The BBT measurement device may be, for example, embodied as an independent device performing only a BBT measurement. Alternatively, the BBT measurement device may be included in a portable device. Non-limiting examples of such portable devices include a personal digital assistant (PDA), a cellular phone, a personal communication service (PCS) phone, a hand-held PC, a Code Division Multiple Access (CDMA)-2000 (1X, 3X) phone, a Wideband CDMA phone, a dual band/dual mode phone, a Global Standard for Mobile Communications (GSM) phone, a mobile broadband system (MBS) phone, a Digital Multimedia Broadcasting (DMB) phone, an MPEG audio layer-3 (MP3) player, a portable multimedia player (PMP), a portable game player, a notebook computer, and the like. In the following description, the BBT measurement device is a portion of such a portable device. However, it is to be understood that this is merely for convenience and that the BBT measuring device need not be a part of a portable device.

Also, menstruation information used throughout the present specification includes a woman's ovulation day information, fertile period information, infertile period information and menstruation period information. In this instance, the menstruation information provided from the portable device may include at least one menstruation information according to judgments of the present invention.

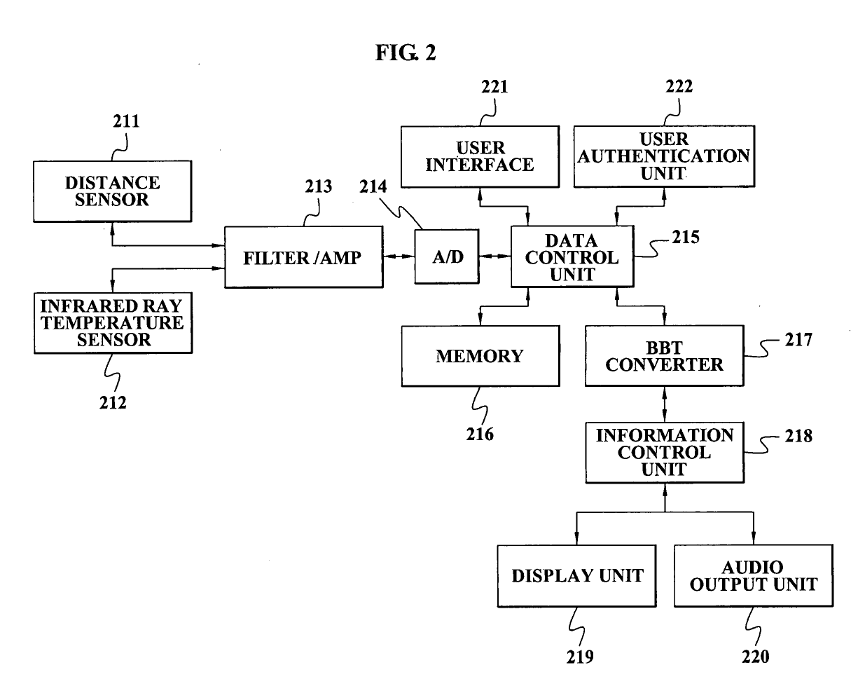

FIG. 2 is a block diagram illustrating a configuration of a portable BBT measurement device according to an embodiment of the present invention.

The portable BBT measurement device according to the present embodiment includes a distance sensor 211, an infrared ray temperature sensor 212, a filter/amplifier 213, an A/D converter 214, a data control unit 215, a memory 216, a BBT converter 217, an information control unit 218, a display unit 219, an audio output unit 220, a user interface 221 and a user authentication unit 222.

The distance sensor 211 measures a distance to at least one ear canal area by emitting a pulse to a user's ear canal. The distance sensor 211 may include any type of sensor capable of measuring a distance, such as an ultrasonic sensor and a laser sensor, by way of non-limiting examples.

When the distance sensor 211 is an ultrasonic sensor, the distance may be measured by emitting an ultrasonic pulse to the user's ear canal via the ultrasonic sensor. Alternatively, when the distance sensor 211 is a laser sensor, the distance may be measured by emitting a laser pulse to the user's ear canal via the laser sensor. In addition, the distance sensor 211 may include various other types of distance measurement sensors which can emit a pulse to a predetermined area and measure the distance to the area where the pulse is emitted.

The infrared ray temperature sensor 212 measures a body temperature in the at least one ear canal area utilizing infrared rays. Specifically, the infrared ray temperature sensor 212 emits a pulse to the user's ear canal, measures a distance to at least one ear canal area and measures a body temperature in the at least one ear canal area utilizing infrared rays. The infrared ray temperature sensor 212 may be an infrared ray sensor which is widely utilized in a field of temperature measurement utilizing infrared rays.

The distance sensor 211 and the infrared ray temperature sensor 212 may be provided around a speaker of the portable device. Specifically, when the user makes a call using the portable device, the portable device may easily detect the user's eardrum location and measure the detected eardrum temperature by installing the distance sensor 211 and the infrared ray temperature sensor 212 around the speaker of the portable device which corresponds to the user's ear. In addition, the distance sensor 211 and the infrared ray temperature sensor 212 may be provided at various locations of the portable device as well as around the speaker of the portable device, to measure the user's eardrum temperature.

The filter/amplifier 213 filters and amplifies an ear canal area distance data signal measured by the distance sensor 211 and an ear canal area temperature data signal measured by the infrared ray temperature sensor 212, for a digital signal conversion of the each signal. The A/D converter 214 converts each of the distance data signal and the temperature data signal, which is an analog signal with respect to the ear canal area, into a digital signal, and transmits the converted digital signal to the data control unit 215.

The data control unit 215 records distance data and body temperature data with respect to the measured at least one ear canal area in the memory 216, to correspond to each other, and recognizes a first body temperature which is recorded in correspondence to a first distance a predetermined standard, among the each distance to the at least one distance data, as the user's eardrum temperature.

Specifically, the data control unit 215 may measure the user' eardrum temperature so as to take the user's exact body temperature. Generally, blood, identical to blood flowing in a hypothalamus of a brain, which adjusts a body temperature, also flows in a human being's eardrum. Accordingly, the eardrum temperature may more accurately reflect the human being's internal body temperature. Also, since the eardrum is positioned in an inwardly deeper location of an ear, the eardrum may not be affected by an external environment and may reflect a more accurate body temperature.

Accordingly, in the present embodiment of the present invention, menstruation information according to the user's BBT may be generated by initially measuring the user's eardrum temperature and converting the eardrum temperature into the BBT. As described above, according to the present embodiment, the menstruation information may be generated by utilizing the eardrum temperature which reflects the user's accurate body temperature. Accordingly, the menstruation information may be more accurately provided for the user.

Ways of reading the eardrum temperature according to the present embodiment may be divided into at least the following two examples based on predetermined operations of the distance sensor 211, the infrared ray temperature sensor 212 and the data control unit 215. Reading of the eardrum temperature according to a first example of the present embodiment is performed in such a manner that the distance sensor 211 detects the user's eardrum location via three distance sensors. Alternatively, reading of the eardrum temperature according to a second example of the present is performed in such a manner that the distance sensor 211 detects the user's eardrum via a signal distance sensor.

Hereinafter, reading of the eardrum temperature by the distance sensor 211, the infrared ray temperature sensor 212 and the data control unit 215 according to these first and second examples of the present embodiment will be described with reference to FIGS. 2 through 6.

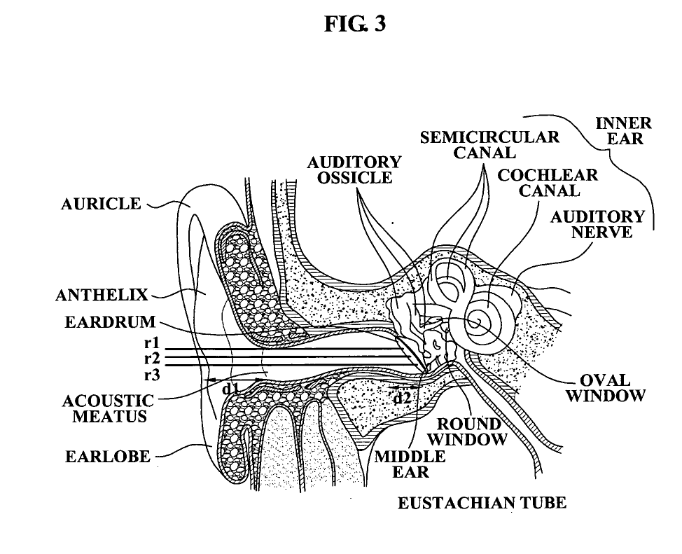

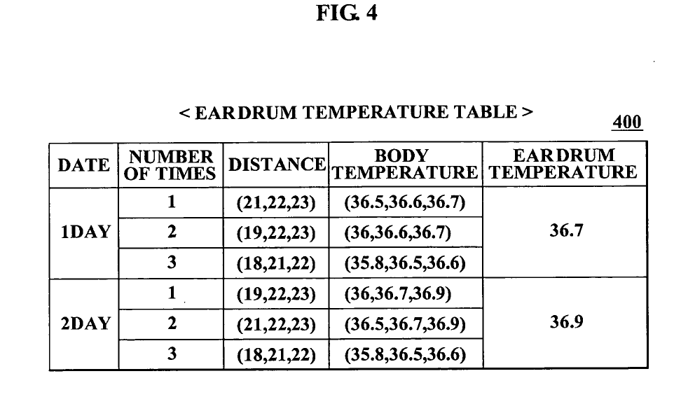

As described above, the operation of reading the eardrum temperature according to the first example may be performed by the distance sensor 211 detecting the user's eardrum location via three distance sensors, which will be described with reference to FIGS. 3 and 4.

FIG. 3 illustrates a user's eardrum and the three distance sensors measuring an eardrum temperature according to the first example of the present embodiment.

As shown in FIG. 3, a human being's ear may include an auricle, an anthelix, an eardrum, an acoustic meatus, an earlobe, a middle ear, an Eustachian tube, an inner ear, an oval window, and a round window. Also, the distance sensor 211 includes a first distance sensor, a second distance and a third distance sensor (not shown).

According to the first example of the present embodiment, the distance sensor 211 measures three sub-distances with respect to a single ear canal area via the three distance sensors. Specifically, the distance sensor 211 may measure a first sub-distance via the first distance sensor, a second sub-distance via the second distance sensor, and a third sub-distance via the third distance sensor, respectively, with respect to the single area canal area.

With the measurement of the each sub-distance, the infrared ray temperature sensor 212 measures a first sub-body temperature in an ear canal area corresponding to the first sub-distance, a second sub-body temperature in an ear canal area corresponding to the second sub-distance and a third sub-body temperature in an ear canal area corresponding to the third sub distance respectively.

The data control unit 215 records the each sub-distance and the each sub-body temperature measured by the distance sensor 211 and the infrared ray temperature sensor 212 respectively, in the memory 216 of the portable device, to correspond to each other. Specifically, the distance and the body temperature data may be recorded in the memory 216, in correspondence to the at least one ear canal area. The distance data may include three sub-distance data and the body temperature data may include three sub-body temperature data.

The data control unit 215 reads distance data which can be recognized as the eardrum temperature, from the at least one distance data which is recorded in the memory 216. The eardrum temperature may be read utilizing a correlation between sub-distances which are included in each distance data.

As shown in FIG. 3, the eardrum is located aslant around the middle ear. Accordingly, the each sub-distance measured by each of the three distance sensors of the distance sensor 211 with respect to the single ear canal area may have a different value. In this instance, when the each sub-distance, i.e. the first distance, the second distance and the third distance are greater than a distance d1 between the each distance sensors and an outer ear, and a variation of the first sub-distance, the second sub-distance and the third sub-distance is smaller than a horizontal distance d2 of the eardrum, the distance data including the first sub-distance, the second sub-distance and the third sub-distance may be recognized as the distance data for reading the eardrum temperature.

Specifically, as shown in FIG. 3, since the eardrum is positioned around the middle ear which is positioned in a deeper area than the outer ear, the each sub-distance must be greater than the distance d1. Also, only when the variation among the sub-distances which is obtained by subtracting a minimum value of the first sub-distance, the second sub-distance and the third sub-distance from a maximum value thereof is smaller than the horizontal distance d2 of the aslant positioned eardrum, the each sub-distance may be determined to have been measured in correspondence to the eardrum.

Accordingly, the data control unit 215 may recognize the distance data which completely satisfies the standard, as the distance data for reading the eardrum temperature. After recognizing the distance data, the data control unit 215 may recognize a sub-body temperature which is recorded in correspondence to a sub-distance having a largest value among the first sub-distance, the second sub-distance, and the third sub-distance, as the user's eardrum temperature.

Specifically, since the sub-distance that has the largest value among the first sub-distance, the second sub-distance, and the third sub-distance reflects a deepest location of the eardrum of the ear canal, the data control unit 215 may recognize the sub-body temperature measured in correspondence to the sub-distance as the user's accurate eardrum temperature, which will be described in detail utilizing an example of an eardrum temperature table as shown in FIG. 4.

FIG. 4 is a table illustrating an example of an eardrum temperature table according to the first example.

As shown in FIG. 4, the distance data and the body temperature data measured by the distance sensor 211 and the infrared ray temperature sensor 212 respectively may be recorded in the memory 216 in a form of an eardrum temperature table 400. As shown in FIG. 4, when a user measures a distance and a body temperature three times in a particular time period during a day, the data control unit 215 reads distance data from the three distance data, so as to measure the eardrum temperature.

As an example, when the distance d1 between the distance sensor and the outer ear is 5 and the horizontal distance d2 of the eardrum is 3, distance data in which all the sub-distances are greater than the d1 and the variation of each sub-distance is smaller than d2 among the distance data corresponding to 1 day in the eardrum table 400 may be (21, 22, 23) of a first time.

Accordingly, the data control unit 215 may read the distance data (21, 22, 23) as the distance data for measuring the eardrum temperature. After this, the data control unit 215 may recognize the third sub-body temperature (36.7 degrees) corresponding to the third sub-distance which has the largest value, 23, among the each sub-distance included in the distance data may be recognized as the user's eardrum temperature.

As described above, the portable device according to the first example of the present embodiment may detect the user's exact eardrum location and measure the temperature of the detected eardrum via three distance sensors comprising distance sensor 211 and the infrared ray temperature sensor 212. Hereinafter, reading of the eardrum of the portable device according to the second example of the present embodiment will be described with reference to FIGS. 2, 5 and 6.

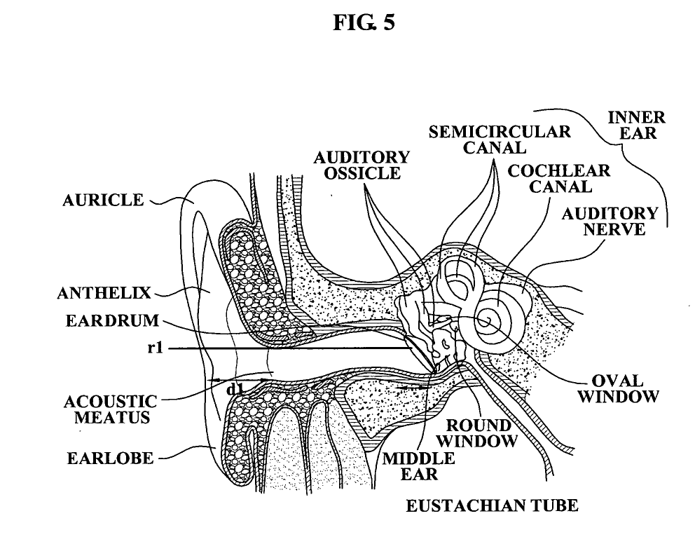

FIG. 5 is a view illustrating a user's eardrum and a single distance sensor measuring an eardrum temperature according to the second example of the present embodiment.

The distance sensor 211 includes a single distance sensor. The distance sensor 211 measures each distance to at least one ear canal area by emitting a pulse to the at least one ear canal area via the single distance sensor. When the distance sensor 211 measures the distance to the at east one ear canal area, the infrared ray temperature sensor 212 measures a body temperature in each ear canal area whose distance is measured.

The data control unit 215 records the measured distance data and the body temperature data in the memory 216 of the portable device, to correspond to each other. The data control unit 215 may recognize a first body temperature corresponding to a first distance which has a largest value from the at least one distance data, as the user's eardrum temperature.

Specifically, the range which can be reached by a pulse that the distance sensor 212 emits to the user's ear canal may be set from the outer ear to the eardrum, which is positioned in the middle ear. Accordingly, an ear canal area which is positioned farthest away from the distance sensor 212 may be recognized as the eardrum. The data control unit 215 recognizes the first body temperature which is recorded in the memory 216 in correspondence to the first distance having the largest distance value, as the user's eardrum temperature.

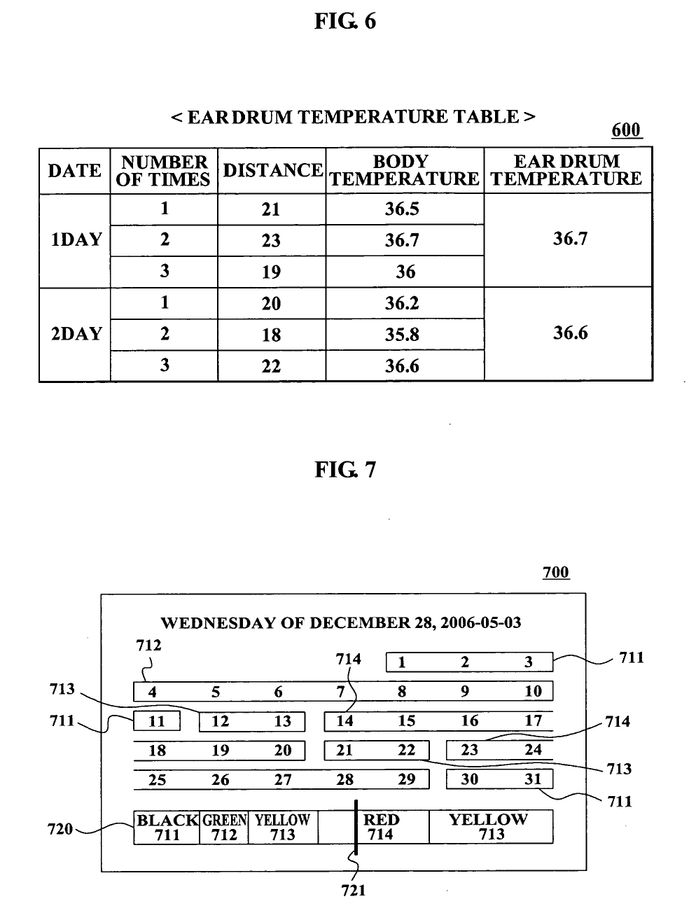

FIG. 6 is a table illustrating an example of an eardrum temperature table according to the second example.

In FIG. 6, an eardrum temperature table 600 records distance data and body temperature data which a user measured with respect to the user's three ear canal areas three times in a particular time period during one day. The data control unit 215 reads distance data of a second time which has the largest value, 23, of the three distance data, as distance data for measuring the user's eardrum temperature. Next, the data control unit 215 may recognize body temperature data, 36.7 degrees, of the second time which is recorded in correspondence to the distance data of the second time, as the user's eardrum temperature.

The user's eardrum temperature which is measured according to the above-described examples with reference to FIGS. 2 through 6 may be converted into the user's BBT by the BBT converter 217, which will be described with reference to FIG. 2.

The BBT converter 217 converts the measured eardrum temperature into the user's BBT and records the converted BBT into the memory 216. The BBT converter 217 accomplished this operation by considering a time period when the user's eardrum temperature is measured and the user's activity state at the time when the user's eardrum temperature is measured, and thus, converts the eardrum temperature into the BBT. This is to calibrate a rise portion of a body temperature according to the measurement time period and the user's activity state.

Generally, a human being's body temperature may be measured to be lowest in the morning and highest in the afternoon. The BBT is usually measured after waking up in the morning. Accordingly, when frequently taking the body temperature, not immediately after waking up, a rise value of the body temperature needs to be calibrated, as the present embodiment does. Also, when the user exercises while taking the body temperature, the body temperature may increase by a predetermined value according to the quantity of motion. Accordingly, the BBT converter 217 may calibrate the rise value of the body temperature according to the user's quantity of motion and output the user's BBT.

Accordingly, the BBT converter 217 may output the BBT according to a time period when the eardrum temperature is measured utilizing the following three equations:
BBT=eardrum temperature−0.09(T−6)−B: 6 hour through 17 hour; [Equation 1]
BBT=eardrum temperature−0.07(30−T)−B: 17 hour through 24 hour; [Equation 2]
and
BBT=eardrum temperature−0.05(6−T)−B: 24 hour through 6 hour. [Equation 3]

Here, −T indicates the measurement time of the eardrum temperature and −B indicates the body temperature rise calibration value according to the user's activity state.

The body temperature rise calibration value according to the user's activity state may be set as a difference value between the eardrum temperature which is measured in the user's each activity state and a first eardrum temperature which is measured when the user lies down, based on the first eardrum temperature. As an example, when the user lies down at 1300 hours on December 7, the user's eardrum temperature may be measured at 36.3 degrees. Also, when the user sits on a chair at 1300 hours on December 5, the user's eardrum temperature may be measured at 37.2 degrees. Also, when the user exercises at 1300 hours on December 6, the user's eardrum temperature may be measured at 37.6 degrees.

In the above-described example, when the body temperature rise calibration value according to the user's activity state of lying down is set as 0.3, the body temperature rise calibration value according to the user's activity state of sitting on a chair may be set as 0.9. Also, the body temperature rise calibration value according to the user's activity state of exercising may be set as 1.3.

The BBT converter 217 may pre-output the body temperature rise calibration value according to the user's activity state by referring to the user's eardrum temperature which is recorded in the memory 216 for a certain period. Specifically, the body temperature rise calibration value may be outputted according to the eardrum temperature which is measured at an identical time, e.g. 1300 hours, for a certain period and the user's each activity state when the eardrum temperature is measured. The BBT converter 217 may frequently output and update the body temperature rise calibration value. Also, as described above, it may be possible to set the body temperature rise calibration value according to the user's activity state of lying down as 0.3 and thus, output the body temperature rise calibration value according to the user's each activity state. However, this is only a non-limiting example and each body temperature rise calibration value may be outputted utilizing various other methods.

Referring to the outputted body temperature rise calibration value according to the user's each activity state, as shown in Equation 1, when the measurement time of the eardrum temperature is any time between 0600 hours and 1700 hours, the BBT converter 217 may subtract 6 from the measurement time, multiply 0.09 and a result of the subtraction and thus, output a first value. Next, the BBT converter 217 may output the BBT by subtracting the first value and the body temperature rise calibration value according to the user's activity state from the eardrum temperature. In this instance, the first value is a value which reflects a change in the body temperature during a day and the body temperature rise calibration value is a value which reflects the user's activity state at the time when the eardrum temperature is measured.

Also, as shown in Equation 2, when the measurement time of the eardrum temperature is any time between 1700 hours and 2400 hours, the BBT converter 217 may subtract the measurement time from 30, multiply 0.07 and a result of the subtraction and thus, output a second value. Next, the BBT converter 217 may output the BBT by subtracting the second value and the body temperature rise calibration value according to the user's activity state from the eardrum temperature. As in Equation 1, the second value is a value which reflects a change in the body temperature during a day and the body temperature rise calibration value is a value which reflects the user's activity state at the time when the eardrum temperature is measured.

Also, as shown in Equation 3, when the measurement time of the eardrum temperature is any time between 2400 hours and 0600 hours, the BBT converter 217 may subtract the measurement time from 6, multiply 0.05 and a result of the subtraction and thus, output a third value. Next, the BBT converter 217 may output the BBT by subtracting the third value and the body temperature rise calibration value according to the user's activity state from the eardrum temperature. As in Equations 2 and 3, the third value is a value which reflects a change in the body temperature during a day and the body temperature rise calibration value is a value which reflects the user's activity state at the time when the eardrum temperature is measured.

As described above, the BBT converter 217 may calibrate the body temperature rise value according to the measurement time of the eardrum temperature and the body temperature rise value according to the user's activity state at the measurement time and thus, output the user's BBT.

The information control unit 218 generates the user's menstruation information from at least one BBT which is recorded in the memory 216 for a predetermined period and controls the generated menstruation information to be displayed or played for the user via the display unit 219 or the audio output unit 220. Specifically, the information control unit 218 may generate the user's menstruation information by referring to the BBT which is recorded for a predetermined period, e.g. a month. In this instance, the menstruation information includes the user's ovulation day information, fertile period information, infertile period information and menstruation period information. The information control unit 218 may display the generated menstruation information to the user via the display unit 219. Also, the information control unit 218 may provide the user with the menstruation information by outputting speech via the audio output unit 220.

FIG. 7 is a diagram illustrating an example of a user's menstruation information displayed on a display unit of a portable device according to an embodiment of the present invention.

Referring to FIGS. 2 and 7, as shown in FIG. 7, the information control unit 218 may display the user's generated menstruation information, e.g. fertile period information, on the display unit 219 of the portable device, i.e. on a screen of the portable device in a form of a calendar. Also, the information control unit 218 may display a graph 720 in which the user's menstruation information is indicated in each color. In the graph 720, a black interval 711 indicates a menstruation period, a green interval 712 indicates an infertile period, a yellow interval 713 indicates a pregnancy possibility period, and a red interval 714 indicates a fertile period.

Each color corresponding to the each interval of the graph 720 may be indicated in the calendar for each date. Specifically, as shown in FIG. 7, when the menstruation period corresponds to 1st through 3rd, 11th and 30th and 31st, the dates may be indicated in black. Also, when the fertile period corresponds to 14th through 20th, and 23rd through 29th, the dates may be indicated in red.

Also, a display bar 721 may be indicated in the graph 720 to show the user's current state. As an example, when today is 18th, today corresponds to the fertile period. Accordingly, the display bar 721 may be indicated in the red interval 714 of the graph 720. Through this, the user may determine the user's current menstruation information, e.g. whether a current day corresponds to the fertile period, from the display screen shown in FIG. 7.

As described above, the information control unit 218 may generate and provide the user's fertile period information from the user's BBT which is measured for a predetermined period. Also, the information control unit 218 may provide the user with an alarm service about the user's menstruation starting day, as one of the menstruation information. Through this, the user may pre-recognize when the menstruation starts and prepare against the menstruation. Also, when the menstruation period is irregular as a result of reading the user's menstruation period for a predetermined period, the information control unit 218 may provide the user with an alarm service, e.g. a hospital visit Also, when the menstruation starting day is not inputted by the user, the information control unit 218 may provide the user with an alarm to induce the user to check for pregnancy.

The user interface 221 receives a selection on the measurement of the BBT from the user. Specifically, only when the selection on the measurement of the BBT is received from the user via the user interface 221, may the data control unit 215 control the distance sensor 211 and the infrared ray temperature sensor 212 to measure the distance to the at least one ear canal and the body temperature in the distance.

The user authentication unit 222 authenticates whether the user who desires to take the BBT is a registered user. Specifically, only when the user authentication unit 222 authenticates the user as the registered user, may the data control unit 215 control the distance sensor 211 and the infrared ray temperature sensor 212 to measure the distance to the ear canal area and the body temperature. Through this, since the measurement of the BBT and the menstruation information according thereto may be provided for only the registered user, the user's privacy may be protected.

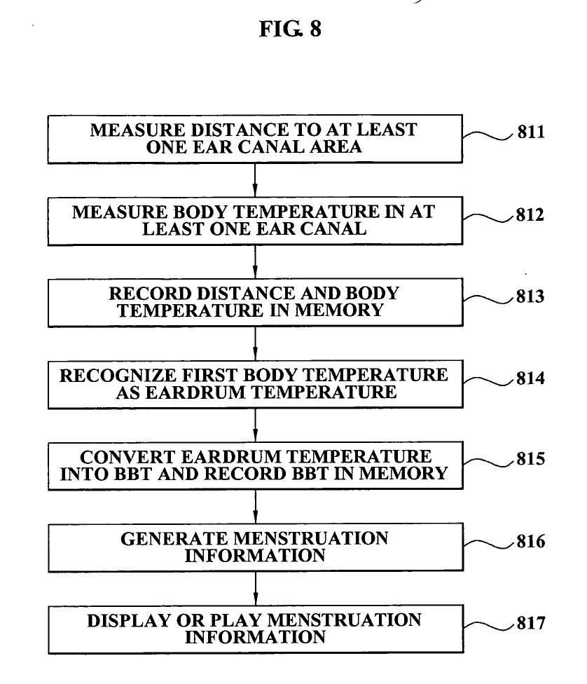

FIG. 8 is a flowchart illustrating a BBT measurement method of a portable device according to an embodiment of the present invention.

In operation 811, the portable device according to the present embodiment measures each distance to at least one ear canal area by emitting a pulse to a user's ear canal. In operation 812, the portable device measures a body temperature in the at least one ear canal area utilizing infrared rays. In operation 813, the portable device records the each distance to the at least one ear canal area and the body temperature in the at least one ear canal area in a predetermined memory, to correspond to each other. In operation 814, the portable device recognizes a first body temperature which is recorded in the memory in correspondence to a first distance satisfying a predetermined standard from the at least one distance, as the user's eardrum temperature.

In operations 811 through 814, the portable device may measure a first sub-distance, a second sub-distance and a third sub-distance corresponding to the at least one ear canal area via a first distance sensor, a second distance sensor and a third distance sensor. Also, the portable device may measure the body temperature in the at least one ear canal area, by measuring a first sub-body temperature in an ear canal area corresponding to the first sub-distance, a second sub-body temperature in an ear canal area corresponding to the second sub-distance, and a third sub-body temperature in an ear canal area corresponding to the third sub-distance. Next, when all the distances of the first sub-distance, the second sub-distance, and the third sub-distance corresponding to the each ear canal area are greater than a distance between the each distance sensor and the user's outer ear and a variation of the first sub-distance, the second sub-distance and the third sub-distance is smaller than a horizontal distance of the eardrum, the portable device may recognize a sub-body temperature corresponding to a sub-distance which has a largest value among the first sub-distance, the second sub-distance, and the third sub-distance, as the user's eardrum temperature.

Alternatively, in operations 811 through 814, the portable device may measure the each distance to the at least one ear canal area via a single distance sensor and also measure the body temperature in the ear canal area corresponding to the each distance. When recognizing the first body temperature as the user's eardrum temperature, the portable device may recognize the first distance which has the largest value among the at least one distance, as the user's eardrum temperature.

In operation 815, the portable device converts the eardrum temperature into the user's BBT and records the converted user's BBT into the memory.

In operation 815, when the measurement time of the eardrum temperature is any time between 0600 hours and 1700 hours, the portable device may subtract 6 from the measurement time, multiply 0.09 and a result of the subtraction and thus, output a first value. Also, the portable device may output the BBT by subtracting the first value and a predetermined calibration value according to the user's current state from the eardrum temperature.

Also, in operation 815, when the measurement time of the eardrum temperature is any time between 1700 hours and 2400 hours, the portable device may subtract the measurement time from 30, multiply 0.07 and a result of the subtraction and thus, output a second value. Also, the portable device may output the BBT by subtracting the second value and a predetermined calibration value according to the user's activity state from the eardrum temperature.

Also, in operation 815, when the measurement time of the eardrum temperature is any time between 2400 hours and 0600 hours, the portable device may subtract the measurement time from 6, multiply 0.05 and a result of the subtraction and thus, output a third value. Also, the portable device may output the BBT by subtracting the third value and the body temperature rise calibration value according to the user's activity state from the eardrum temperature.

The portable device may set the predetermined calibration value according to the user's current state as a difference value between an eardrum temperature which is measured in the user's each activity state and a first eardrum temperature which is measured when the user lies down, based on the first eardrum temperature.

In operation 816, the portable device generates the user's menstruation information from at least one BBT which is recorded in the memory for a predetermined period. In operation 817, the portable device controls the generated menstruation information to be displayed or played for the user via the display unit or the audio output unit of the portable device.

The BBT measurement method of the portable device according to the present embodiment which has been described with reference to FIG. 8 may include all the operations according to the configuration of the Portable BBT measurement device which has been described with reference to FIGS. 2 through 7.

According to the above-described embodiments of the present invention, there is provided a portable BBT measurement device and method which can convert a measured body temperature into a BBT considering a user's current state and measurement time, so that the user may easily measure the user's BBT without regard to a time and a place.

According to the above-described embodiments of the present invention, there is provided a portable BBT measurement device and method which can detect a location of eardrum of a user via a distance sensor, such as an ultrasonic sensor, a laser sensor, etc., measure a temperature of the detected eardrum via an infrared ray temperature sensor, so that the user may measure the user's internal temperature without regard to a change in external environments.

According to the above-described embodiments of the present invention, there is provided a portable BBT measurement device and method which enables a user of the portable device to easily take the user's temperature while the user is making a call by installing a distance sensor and an infrared ray temperature sensor around a speaker of the portable device.

According to the above-described embodiments of the present invention, there is provided a portable BBT measurement device and method which can record a user's measured BBT in a predetermined memory, generate the user's fertile period information, infertile period information, ovulation day information, or menstruation period information from the BBT that is recorded for a predetermined period, e.g. a month, and provide the user with the generated menstruation information, so that the user may accurately know her own menstruation information with only a measurement of a body temperature.

According to the above-described embodiments of the present invention, there is provided a portable BBT measurement device and method which can protect a user's privacy by measuring the user's temperature and generating the menstruation information only when the user is authenticated as a subject for measurement of a BBT via speech recognition or when a selection is received from the user.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

What is claimed is:

1. A method of measuring a basal body temperature, the method comprising:
measuring at least one distance to at least one ear canal area;
measuring a body temperature in the at least one ear canal area;
recognizing a first body temperature corresponding to a first distance which satisfies a predetermined standard, among the measured at least one distance to the at least one ear canal area, as the user's eardrum temperature; and
converting the eardrum temperature into the user's basal body temperature.

2. The method of claim 1, wherein:
in the measuring at least one distance, measures of a first sub-distance, a second sub-distance and a third sub-distance corresponding to the at least one ear canal area are respectively made via a first distance sensor, a second distance sensor and a third distance sensor,
in the measuring a body temperature, measures of a first sub-body temperature in an ear canal area corresponding to the first sub-distance, a second sub-body temperature in an ear canal area corresponding to the second sub-distance and a third sub-body temperature in an ear canal area corresponding to the third sub-distance are made, and
in the recognizing a first body temperature, a sub-body temperature corresponding to a sub-distance which has a largest value among the first sub-distance, the second sub-distance, and the third sub-distance, is recognized as the user's eardrum temperature, when all the distances of the first sub-distance, the second sub-distance and the third sub-distance corresponding to the at least one ear canal area are greater than a distance between each distance sensor and the user's outer ear and a variation of the first sub-distance, the second sub-distance and the third sub-distance being smaller than a horizontal distance to the eardrum.

3. The method of claim 1, wherein:
in the measuring at least one distance, the at least one distance to the at least one ear canal area is measured via a single distance sensor,
in the measuring a body temperature, the body temperature in the ear canal area corresponding to the each distance is measured, and
in the recognizing a first body temperature, the first body temperature corresponding to the first distance which has a largest value among the at least one distance is recognized as the user's eardrum temperature.

4. The method of claim 1, wherein the converting the eardrum temperature includes:
reading a time when the eardrum temperature is measured;
reading an activity state of the user at the time when the eardrum temperature is measured; and
converting the eardrum temperature into the basal body temperature based on either the user's body temperature rise value according to the read time or a predetermined body temperature calibration value according to the read activity state.

5. A method of measuring a basal body temperature, the method comprising:
measuring a user's body temperature;
reading a time when the body temperature is measured; and
converting the measured body temperature into the basal body temperature based on the user's body temperature rise value according to the read time.

6. The method of claim 5, further comprising:
reading the user's activity state at the time when the body temperature is measured; and
converting the measured body temperature into the basal body temperature based on a predetermined body temperature calibration value according to the read activity state.

7. A method of measuring a basal body temperature, the method comprising:
measuring a user's body temperature;
reading the user's activity state at a time when the body temperature is measured; and
converting the measured body temperature into the basal body temperature based on a predetermined body temperature calibration value according to the read activity state.

8. The method of claim 7, wherein the converting the measured body temperature is based on a difference value between a first body temperature which is measured when the user lies down and a body temperature which is measured in the user's each activity state, as the body temperature calibration value, based on the first body temperature.

9. The method of claim 7, further comprising:
reading a time when the body temperature is measured; and
converting the measured body temperature into the basal body temperature based on the user's body temperature rise value according to the read time.

10. A method of measuring a basal body temperature, the method comprising:
measuring at least one distance to at least one ear canal area;
measuring a body temperature in the at least one ear canal area;
recording the measured at least one distance and the measured body temperature in a memory, to correspond to each other;
recognizing a first body temperature which is measured in correspondence to a first distance satisfying a predetermined standard, among the measured at least one distance to the at least one ear canal area, as the user's eardrum temperature;
converting the eardrum temperature into the basal body temperature based on either the user's body temperature rise value according to a time when the eardrum temperature is measured or a predetermined body temperature calibration value according to the user's activity state at the time when the eardrum temperature is measured, and recording the converted basal body temperature into the memory; and
generating the user's menstruation information from at least one basal body temperature which is recorded in the memory for a predetermined period and controlling the generated menstruation information to be displayed or played for the user.

11. The method of claim 10, wherein the menstruation information includes the user's ovulation day information, fertile period information, infertile period information and menstruation period information.

12. A computer-readable recording medium storing a program for implementing a method of measuring a basal body temperature, the method comprising:
measuring at least one distance to at least one ear canal area;
measuring a body temperature in the at least one ear canal area;
recognizing a first body temperature corresponding to a first distance which satisfies a predetermined standard, among the at least one measured distance to the at least one ear canal area, as the user's eardrum temperature; and
converting the eardrum temperature into the user's basal body temperature.

13. A basal body temperature measurement device comprising:
a distance sensor measuring at least one distance to at least one ear canal area by emitting a pulse to a user's ear canal;
an infrared ray temperature sensor measuring a body temperature in the at least one ear canal area utilizing infrared rays;
a data control unit recording the measured at least one distance and the measured body temperature in a memory, to correspond to each other, and recognizing a first body temperature which is measured in correspondence to a first distance satisfying a predetermined standard, among the at least one distance to the at least one ear canal area, as the user's eardrum temperature;
a basal body temperature converter converting the eardrum temperature into the basal body temperature based on either the user's body temperature rise value according to a time when the eardrum temperature is measured or a predetermined body temperature calibration value according to the user's activity state at the time when the eardrum temperature is measured, and recording the converted basal body temperature into the memory; and
an information control unit generating the user's menstruation information from at least one basal body temperature which is recorded in the memory for a predetermined period and controlling the generated menstruation information to be displayed or played for the user via a display unit or an audio output unit.

14. The basal body temperature measurement device of claim 13, further comprising a user interface receiving a selection on measurement of the basal body temperature from a user,
wherein the data control unit controls the distance sensor and the infrared ray temperature sensor to measure a distance to the at least one ear canal area and the body temperature, when the selection on the measurement of the basal body temperature is received from the user.

15. The basal body temperature measurement device of claim 13, further comprising a user authentication unit authenticating whether the user is a registered user,
wherein the data control unit controls the distance sensor and the infrared ray temperature sensor to measure the at least one distance to the at least one ear canal area and the body temperature, when the user authentication unit authenticates the user as the registered user.

16. An apparatus for measuring a basal body temperature, comprising:
a distance sensor measuring distances to ear canal areas;
a temperature sensor measuring body temperatures in the ear canal areas corresponding to the measured distances;
determining a body temperature corresponding to a first one of the measured distances satisfying set criteria, as the user's eardrum temperature; and
converting the eardrum temperature into the basal body temperature.
